(19) United States
(12) Patent Application Publication
(10) Pub. No.: US 2024/0139261 A1
(43) Pub. Date: May 2, 2024

(54) COMPOSITION FOR IMPROVING GUT MICROBIOTA AND METHOD FOR IMPROVING GUT MICROBIOTA WITH THE COMPOSITION THEREOF

(71) Applicant: TCI CO., LTD., Taipei (TW)

(72) Inventors: YUNG-HSIANG LIN, Taipei (TW); CHU-HAN HUANG, Taipei (TW); YI-LIN CHEN, Taipei (TW)

(21) Appl. No.: 18/051,017

(22) Filed: Oct. 31, 2022

(51) Int. Cl. A61K 35/747; A61K 35/742; A61K 35/745; A61P 1/14

(52) U.S. Cl. CPC A61K 35/747; A61K 35/742; A61K 35/745; A61P 1/14

(57) ABSTRACT

Provided is a composition for improving gut microbiota, including: a bacterial species combination consisting of Limosilactobacillus fermentum TCI275 with an accession number of BCRC 910940, Bifidobacterium animalis subsp. lactis TCI604 with an accession number of BCRC 910887, and Weizmannia coagulans TCI803 with an accession number of BCRC 910946. Provided is a method for improving gut microbiota of a subject in need thereof with the bacterial species combination.

### REFERENCE OF AN ELECTRONIC SEQUENCE LISTING

The contents of the electronic sequence listing (P223665USI_ST26.xml; Size: 4 KB; and Date of Creation: Mar. 22, 2023) is herein incorporated by reference in its entirety.

### BACKGROUND

### Technical Field

The present disclosure relates to a composition for gastrointestinal health, and in particular, to a composition for improving gut microbiota and a method for improving gut microbiota with the same.

### Related Art

It is currently known that causes of the imbalance of gut microbiota roughly include a congenital cause and an acquired cause. The congenital cause refers to genetic inheritance. The acquired cause includes dietary habits, life stress, and use of antibiotics.

The imbalance of gut microbiota often causes diseases. Common diseases include digestive tract-related symptoms, reduction of immunity, allergies, obesity, depression, and dementia.

Studies have shown that consumption of dietary fiber and fermented foods, and supplementation of probiotics are beneficial to healthy growth of gut microbiota.

Therefore, how to screen probiotic populations for a probiotic combination for improving gut microbiota will be a topic that all researchers need to resolve.

### SUMMARY

In view of this, in some embodiments, a composition for improving gut microbiota is provided, including: a bacterial species combination consisting of Limosilactobacillus fermentum TCI275 with an accession number of BCRC 910940, Bifidobacterium animalis subsp. lactis TCI604 with an accession number of BCRC 910887, and Weizmannia coagulans TCI803 with an accession number of BCRC 910946.

In some embodiments, use of a bacterial species combination for preparing a composition for improving gut microbiota is provided. The bacterial species combination consists of the bacteria in the above embodiments.

In some embodiments, a method for improving gut microbiota of a subject in need thereof with a bacterial species combination is provided, including administering to the subject an effective dose of a composition consisting of Limosilactobacillus fermentum TCI275 with an accession number of BCRC 910940, Bifidobacterium animalis subsp. lactis TCI604 with an accession number of BCRC 910887, and Weizmannia coagulans TCI803 with an accession number of BCRC 910946.

Based on the above, in the composition for improving gut microbiota according to any one of the embodiments, the bacterial species combination consisting of the specific bacterial species has the effect of improving gut microbiota of a subject. In some embodiments, the bacterial species combination consisting of the specific bacterial species has an effect of increasing an abundance of at least one of good bacteria. In some embodiments, the bacterial species combination consisting of the specific bacterial species has an effect of increasing a content of short-chain fatty acids (SCFAs). Therefore, the bacterial species combination is suitable for preparing a composition for improving gut microbiota. The bacterial species combination is suitable for a method for improving gut microbiota of a subject in need thereof.

### BRIEF DESCRIPTION OF THE DRAWINGS

FIG. 1 is a bar chart showing relative gas production rates of bacterial species combinations according to an embodiment.

FIG. 2 is a bar chart showing relative comprehensive somatosensory assessment value of abdominal bloating with the bacterial species combination according to an embodiment.

FIG. 3A is a bar chart showing relative severity of difficulty in burping with the bacterial species combination according to an embodiment.

FIG. 3B is a bar chart showing relative severity of feeling of abdomen tied in knots with the bacterial species combination according to an embodiment.

FIG. 3C is a bar chart showing relative severity of significant abdominal bulge with the bacterial species combination according to an embodiment.

FIG. 4 is a bar chart showing relative comprehensive somatosensory assessment value of defecation status with the bacterial species combination according to an embodiment.

FIG. 5 is a bar chart showing defecation frequency with the bacterial species combination according to an embodiment.

FIG. 6A is a bar chart showing the relative abundance (of Akkermansia bacteria) with the bacterial species combination according to an embodiment.

FIG. 6B is a bar chart showing the relative abundance (of Ruminococcaceae bacteria) with the bacterial species combination according to an embodiment.

FIG. 6C is a bar chart showing the relative abundance (of Bilophila bacteria or Veillonella bacteria) with the bacterial species combination according to an embodiment.

FIG. 7A is a bar chart showing the relative content of acetic acid with the bacterial species combination according to an embodiment.

FIG. 7B is a bar chart showing the relative content of propionic acid with the bacterial species combination according to an embodiment.

FIG. 7C is a bar chart showing the relative content of isobutyric acid with the bacterial species combination according to an embodiment.

### DETAILED DESCRIPTION

In order to enable a person of ordinary skill in the art to understand the characteristics of the present invention, the following general descriptions and definitions are given for the terms mentioned in the specification and the scope of the patent application. Unless otherwise specified, all technical and scientific terms used herein have the meanings as commonly understood by a person skilled in the art to the present invention. In case of conflict, the definitions in this specification shall control.

Herein, the term “subject” refers to a human or a non-human mammal, preferably a human.

In some embodiments, a bacterial species combination of compound probiotics consists of Limosilactobacillus fermentum TCI275, Bifidobacterium animalis subsp. lactis TCI604, and Weizmannia coagulans TCI803. The Limosilactobacillus fermentum TCI275 is deposited at the Food Industry Research and Development Institute with an accession number of BCRC 910940 and deposited at the Deutsche Sammlung von Mikroorganismen and Zellkulturen GmbH (DSMZ) with an accession number of DSM 33289. The Bifidobacterium animalis subsp. lactis TCI604 is deposited at the Food Industry Research and Development Institute with an accession number of BCRC 910887 and deposited at the DSMZ with an accession number of DSM 33303. The Weizmannia coagulans TCI803 is deposited at the Food Industry Research and Development Institute with an accession number of BCRC 910946 and deposited at the DSMZ with an accession number of DSM 33486.

In some embodiments, the Limosilactobacillus fermentum TCI275 and the Bifidobacterium animalis subsp. lactis TCI604 are isolated from human breast milk. In some embodiments, the Weizmannia coagulans TCI803 is isolated from orange peels.

In an embodiment, the Limosilactobacillus fermentum TCI275, the Bifidobacterium animalis subsp. lactis TCI604, and the Weizmannia coagulans TCI803 in the bacterial species combination may be active strains. In another embodiment, the Limosilactobacillus fermentum TCI275, the Bifidobacterium animalis subsp. lactis TCI604, and the Weizmannia coagulans TCI803 in the bacterial species combination may be deactivated strains.

In some embodiments, the Limosilactobacillus fermentum TCI275, the Bifidobacterium animalis subsp. lactis TCI604, and the Weizmannia coagulans TCI803 in the bacterial species combination are in a ratio of 1:1:1.

In some embodiments, the bacterial species combination of the compound probiotics is suitable for preparing a composition for improving gut microbiota. In other words, the composition for improving gut microbiota includes a bacterial species combination consisting of Limosilactobacillus fermentum TCI275 with an accession number of BCRC 910940 (same as the strain with an accession number of DSM 33289), Bifidobacterium animalis subsp. lactis TCI604 with an accession number of BCRC 910887 (same as the strain with an accession number of DSM 33303), and Weizmannia coagulans TCI803 with an accession number of BCRC 910946 (same as the strain with an accession number of DSM 33486).

In some embodiments, the composition for improving gut microbiota may be a food composition. That is, the food composition contains an effective content of the bacterial species combination.

In some embodiments, the food composition containing Limosilactobacillus fermentum TCI275, Bifidobacterium animalis subsp. lactis TCI604, and Weizmannia coagulans TCI803 is a health food, a food for special health use, a functional food, a nutritional supplement, or a special nutritional food, but is not limited thereto.

In some embodiments, the health food, food for special health use, functional food, nutritional supplement, or special nutritional food may be consumed at different frequencies, such as once a day, multiple times a day, or once every few days, depending on the age, weight, and health status of a subject who needs the administration of the food composition. Alternatively, the contents of the Limosilactobacillus fermentum TCI275, Bifidobacterium animalis subsp. lactis TCI604, and Weizmannia coagulans TCI803 in the health food, food for special health use, functional food, nutritional supplement, or special nutritional food in any one of the embodiments may be adjusted according to the needs of subjects who need the administration of the food composition, for example, to the daily dose.

In some embodiments, the food composition containing the bacterial species combination may further include an adjuvant. For example, the adjuvant may be maltodextrin, malic acid, sucralose, citric acid, fruit spice, honey spice, steviol glycoside, or a combination thereof. The type and quantity of selected carriers fall within the scope of professionalism and routine technology of those skilled in the art.

### Example 1: Analysis on Gas Production by Fermentation of Carbohydrate In Vitro

According to previous research, one of the causes of abdominal bloating is overgrowth of bad bacteria and gas production by fermentation in the gut.

### 1-1. Materials

(1) Durham fermentation tube: for trapping gases produced by proliferated bacteria. A bacterial strain to be tested was cultured in a medium-containing test tube with a Durham fermentation tube having a small inner diameter inverted inside. If part of the medium inside the Durham fermentation tube is replaced by air bubbles, it is determined that there is gas produced by the bacterial strain.

(2) MRS medium (BD™ Difco™ Lactobacilli MRS Broth), purchased from Gibco, No. 12100-046.

(3) Bacterial strains for testing: as shown in Table 1.

TABLE 1
Bacterial strains for testing

| | Taiwan/International | Initial bacterial |
|---|---|---|
| Bacterial strains for testing | accession number | broth |
| Bifidobacterium animalis subsp. lactis TCI604 | BCRC 910887/DSM | TCI604 broth |
| | 33303 | |
| Bifidobacterium longum subsp. longum TCI068 | DSM 28119 | TCI068 broth |
| Weizmannia coagulans TCI803 | BCRC 910946/DSM | TCI803 broth |
| | 33486 | |
| Limosilactobacillus fermentum TCI275 | BCRC 910940/DSM | TCI275 broth |
| | 33289 | |
| Lacticaseibacillus paracasei TCI058 | BCRC 910882/DSM | TCI058 broth |
| | 33286 | |
| Leuconostoc mesenteroides subsp. mesenteroides | BCRC 910981/DSM | TCI007 broth |
| TCI007 | 33502 | |
| Lactococcus lactis subsp. lactis TCI333 | BCRC 911143/DSM | TCI333 broth |
| | 34375 | |
| E. coli | ATCC 23815 | E. coli broth |

1-2. Experimental Procedure

(1) Each bacterial strain for testing (as shown in Table 1) that had been cryopreserved in glycerol was activated by the following activation process. Activation process: Cryogenic tubes containing the above bacterial strains were warmed, and the warmed bacterial strains were inoculated into fresh MRS media to culture in an anaerobic environment at 37° C. for 24 h, to obtain activated bacterial broths, as shown in Table 1. Then, the bacterial counts of the activated bacterial broths were adjusted to obtain initial bacterial broths with an O.D.600 value of 1. The O.D.600 value is the absorbance measured by an ELISA Reader at a wavelength of 600 nm.

(2) Each initial bacterial broth was inoculated into a test tube, with a Durham fermentation tube inverted inside, containing an appropriate amount of MRS medium. The groups and inoculum sizes are shown in Table 2. The group in which E. coli (as a pathogen) was inoculated alone is a positive control group. In each experimental group, a ratio of the inoculum size (Ill) of the pathogen to the inoculum size (Ill) of the probiotic was 1:1. In this example, in the case of a total inoculum size of the bacterial broth of 200 μl, the inoculum size of the pathogen and the inoculum size of the probiotic were both 100 μl (as an original inoculum size of the probiotic). In the experimental groups 4 to 9, compound probiotics were inoculated, in which each probiotic had an equal proportion of the original inoculum size.

TABLE 2
Group and inoculum size of each group

| Group | Inoculum size |
|---|---|
| Blank group | 200 μl of MRS medium with no bacteria |
| Positive control group | 200 μl of E. coli broth |
| Experimental group 1 | E. coli broth:TCI275 broth = 100 μl:100 μl |
| (E. coli + TCI275) | |
| Experimental group 2 | E. coli broth:TCI604 broth = 100 μl:100 μl |
| (E. coli + TCI604) | |
| Experimental group 3 | E. coli broth:TCI803 broth = 100 μl:100 μl |
| (E. coli + TCI803) | |
| Experimental group 4 | E. coli broth:TCI007 broth:TCI068 broth:TCI333 |
| (E. coli + TCI007 + | broth = 100 μl:33.3 μl:33.3 μl:33.3 μl |
| TCI068+ TCI333) | |
| Experimental group 5 | E. coli broth:TCI007 broth:TCI058 broth:TCI275 |
| (E. coli + TCI007 + | broth = 100 μl:33.3 μl:33.3 μl:33.3 μl |
| TCI058 + TCI275) | |
| Experimental group 6 | E. coli broth:TCI007 broth:TCI064 broth:TCI803 |
| (E. coli + TCI007 + | broth = 100 μl:33.3 μl:33.3 μl:33.3 μl |
| TCI064 + TCI803) | |
| Experimental group 7 | E. coli broth:TCI007 broth:TCI275 broth:TCI604 |
| (E. coli + TCI007 + | broth = 100 μl:33.3 μl:33.3 μl:33.3 μl |
| TCI275 + TCI604) | |
| Experimental group 8 | E. coli broth:TCI058 broth:TCI604 broth:TCI803 |
| (E. coli + TCI058 + | broth = 100 μl:33.3 μl:33.3 μl:33.3 μl |
| TCI604 + TCI803) | |
| Experimental group 9 | E. coli broth:TCI275 broth:TCI604 broth:TCI803 |
| (E. coli + TCI275 + | broth = 100 μl:33.3 μl:33.3 μl:33.3 μl |
| TCI604 + TCI803) | |

(3) The inoculated bacteria in the test tubes were cultured in an anaerobic environment at 37° C. for 3 days, and then the amount of gas, produced due to bacterial proliferation, collected in the Durham fermentation tube was observed (that is, whether the culture broth was emptied out of the Durham fermentation tube from the top was observed), and was quantified to obtain the corresponding emptying length. The relative gas production rate of each experimental group was calculated based on the emptying length of the positive control group by Formula 1.

$$Relative gas production rate ( \% ) = \frac{( Emptying length of each experimental group )}{( Emptying length of positive control group )} \times 100 \qquad Formula 1$$

1-3. Experimental Results

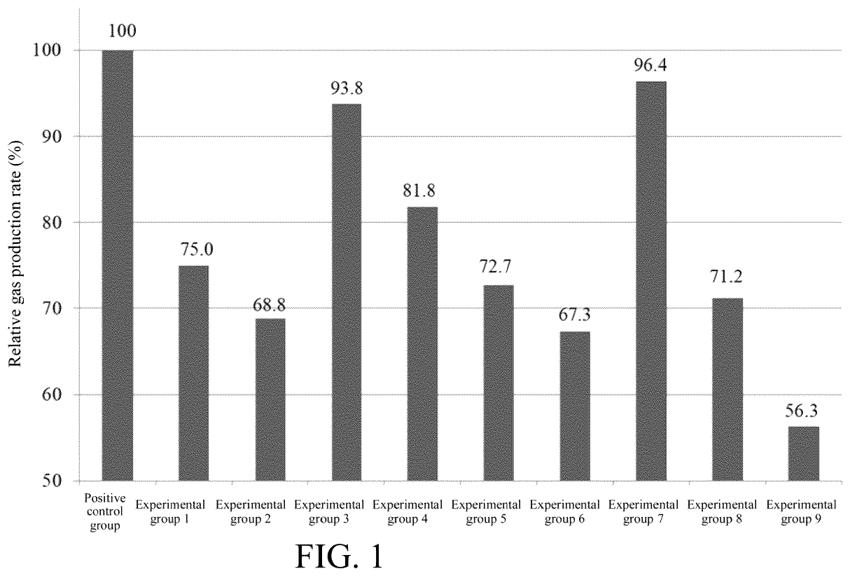

FIG. 1 was a bar chart showing the relative gas production rate of each group, and the value of the relative gas production rate of each group was shown in Table 3. Herein, the Durham fermentation tube was not emptied in the blank group, so it could be determined that no contamination of non-colonizing strains occurred during the experiment.

TABLE 3
Relative gas production rate of each group

| | Gas |
|---|---|
| | production |
| Group | rate (%) |
| Positive control group | 100 |
| Experimental group 1 (E. coli + TCI275) | 75.0 |
| Experimental group 2 (E. coli + TCI604) | 68.8 |
| Experimental group 3 (E. coli + TCI803) | 93.8 |
| Experimental group 4 (E. coli + TCI007 + TCI068 + TCI333) | 81.8 |
| Experimental group 5 (E. coli + TCI007 + TCI058 + TCI275) | 72.7 |
| Experimental group 6 (E. coli + TCI007 + TCI064 + TCI803) | 67.3 |
| Experimental group 7 (E. coli + TCI007 + TCI275 + TCI604) | 96.4 |
| Experimental group 8 (E. coli + TCI058 + TCI604 + TCI803) | 71.2 |
| Experimental group 9 (E. coli + TCI275 + TCI604 + TCI803) | 56.3 |

Referring to FIG. 1 and Table 3, compared with a single probiotic (experimental groups 1 to 3), the gas production rate of experimental group 9 was significantly reduced. In other words, the gas production rate of experimental group 1 was reduced by 25%, the gas production rate of experimental group 2 was reduced by 31.2%, the gas production rate of experimental group 3 was reduced by 6.2%, and the gas production rate of experimental group 9 was significantly reduced by 43.7%. In addition, compared with the compound probiotics in other experimental groups (experimental groups 4 to 8), the gas production rate of experimental group 9 was also reduced.

It could be learned that the bacterial species combination of the compound probiotics in experimental group 9 could inhibit the growth of E. coli and reduce the fermentation of carbohydrates by E. coli, to reduce the gas produced due to the proliferation of pathogens (some of them are classified as enteropathogenic E. coli), which facilitated alleviating abdominal bloating of subjects.

### Example 2: Preparation of Compound Probiotics Powder

According to the relative gas production rate of experimental group 9 in Example 1, the bacterial species combination (consisting of Limosilactobacillus fermentum TCI275, Bifidobacterium animalis subsp. lactis TCI604, and Weizmannia coagulans TCI803) of the compound probiotics was selected and prepared into a compound probiotics powder (viable bacteria powder) of this bacterial species combination. In addition, the preparation of bacterial powder is well known to those of ordinary skill in the art, and details are not described herein again.

### Example 3: Human Subject Experiment

Experimental dose: 100 mg of compound probiotics powder in Example 2/day. In the 100 mg of compound probiotics powder in Example 2, the content of Limosilactobacillus fermentum TCI275 was 3×10⁹ CFU, the content of Bifidobacterium animalis sub sp. lactis TCI604 was 3×10⁸ CFU, and the content of Weizmannia coagulans TCI803 was 9×10⁸ CFU.

Experimental process design: The experiment was carried out with eight subjects by self-control. Each subject took one capsule containing 100 mg of compound probiotics powder in Example 2 every day for four weeks. The day before administration was regarded as week 0 (WO), the day after administration for 2 weeks was regarded as week 2 (W2), and the day after administration for 4 weeks was regarded as week 4 (W4). The eight subjects were adults under the age of 65 suffering from gastrointestinal discomfort, defecation discomfort, constipation, and abdominal bloating.

Test items: Comprehensive somatosensory assessment related to the gastrointestinal tract, survey on alleviation of defecation discomfort and promotion of defecation with a questionnaire, detection of gut microbiota, and detection of content of short-chain fatty acids (SCFAs).

It is to be noted that the test items were carried out separately on the same batch of subjects.

3-1. Analysis on Compound Probiotics in Alleviating Gastrointestinal Discomfort

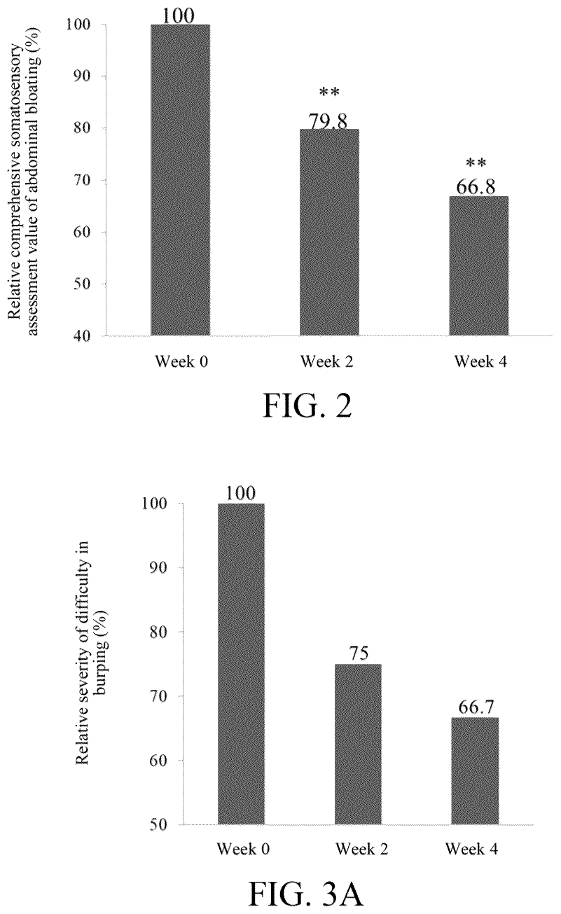

The alleviation of the subjects was recorded with a questionnaire, and the comprehensive somatosensory assessment of abdominal bloating was used to analyze the effect of the compound probiotics on the alleviation of abdominal bloating. Specifically, the survey on comprehensive somatosensory assessment of abdominal bloating was carried out with a questionnaire at week 0, week 2, and week 4 separately. The questionnaire and scoring method are shown in Table 4. The left column of Table 4 lists various symptoms for assessment, including burping, farting, and severe tingling or cramping in the lower abdomen. The first row of Table 4 lists the scoring method, where a higher score indicates a more significant symptom (more severe). For example, if the subject burped very severely, “completely agreed” (for 7 points) would be checked. On the contrary, if the subject did not burp, “completely disagreed” (for 1 point) would be checked. In addition, the statistical results of Table 4 are shown in Table 5. The relative comprehensive somatosensory assessment values (%) of week 2 and week 4 were calculated respectively based on the mean value of week 0, as shown in FIG. 2.

TABLE 4
Questionnaire for comprehensive somatosensory assessment of
abdominal bloating

| | Completely | | Partially | | Partially | | Completely |
|---|---|---|---|---|---|---|---|
| | disagreed | Disagreed | disagreed | Fair | agreed | Agreed | agreed |
| Symptom | (1 point) | (2 points) | (3 points) | (4 points) | (5 points) | (6 points) | (7 points) |
| Belching; | | | | | | | |
| burping | | | | | | | |
| Difficulty | | | | | | | |
| in burping | | | | | | | |
| (wanted to | | | | | | | |
| burp but | | | | | | | |
| failed) | | | | | | | |
| Farting | | | | | | | |
| Severe | | | | | | | |
| tingling or | | | | | | | |
| cramping | | | | | | | |
| in lower | | | | | | | |
| abdomen | | | | | | | |
| Feeling of | | | | | | | |
| abdomen | | | | | | | |
| “tied in | | | | | | | |
| knots” | | | | | | | |
| Abdominal | | | | | | | |
| bloating | | | | | | | |
| (swelling) | | | | | | | |
| and | | | | | | | |
| tightness | | | | | | | |
| Significant | | | | | | | |
| abdominal | | | | | | | |
| bulge | | | | | | | |
| Loss of | | | | | | | |
| appetite | | | | | | | |

TABLE 5
Statistical results of comprehensive somatosensory
assessment of abdominal bloating

| | Score of each item | | |
|---|---|---|---|
| | (sum of scores of 8 subjects) | | |
| Symptom | Week 0 | Week 2 | Week 4 |
| Belching; burping | 30 | 23 | 25 |
| Difficulty in burping (wanted to burp but | 24 | 18 | 16 |
| failed) | | | |
| Farting | 37 | 27 | 28 |
| Severe tingling or cramping in lower | 21 | 20 | 17 |
| abdomen | | | |
| Feeling of abdomen tied in knots | 19 | 17 | 12 |
| Abdominal bloating (swelling) and | 29 | 23 | 13 |
| tightness | | | |
| Significant abdominal bulge | 29 | 22 | 14 |
| Loss of appetite | 19 | 16 | 14 |
| Mean | 26.0 | 20.8 | 17.4 |
| % | 100.0 | 79.8 | 66.8 |

Referring to FIG. 2, compared with week 0, the relative comprehensive somatosensory assessment value of abdominal bloating at week 2 was reduced to 79.8% (** represents a very significant statistical difference compared with week 0 (p<0.01)). In addition, compared with week 0, the relative comprehensive somatosensory assessment value of abdominal bloating at week 4 was reduced to 66.8% (** represents a very significant statistical difference compared with week 0 (p<0.01)). Based on this, after the subjects took the compound probiotics powder for four weeks, the relative comprehensive somatosensory assessment value of abdominal bloating of the subjects decreased continuously. It could be learned that the bacterial species combination of the compound probiotics had the effect of alleviating abdominal bloating of the human body.

In addition, in Table 5, the questionnaire results of difficulty in burping (wanted to burp but failed), feeling of abdomen tied in knots, and significant abdominal bulge were analyzed respectively, that is, relative to week 0, the total scores of the above symptoms of the eight subjects at week 2 and week 4 were converted into percentages respectively, as shown in Table 6.

TABLE 6
Statistical results of difficulty in burping
(wanted to burp but failed), feeling of abdomen
tied in knots, and significant abdominal bulge

| | | | | Percentages | | |
|---|---|---|---|---|---|---|
| | Total scores of 8 | | | converted from | | |
| | subjects (score) | | | total scores (%) | | |
| | Week | Week | Week | Week | Week | Week |
| Symptom | 0 | 2 | 4 | 0 | 2 | 4 |
| Difficulty in | 24 | 18 | 16 | 100.0 | 75.0 | 66.7 |
| burping (wanted to | | | | | | |
| burp but failed) | | | | | | |
| Feeling of abdomen | 19 | 17 | 12 | 100.0 | 89.5 | 63.2 |
| tied in knots | | | | | | |
| Significant | 29 | 22 | 14 | 100.0 | 75.9 | 48.3 |
| abdominal bulge | | | | | | |

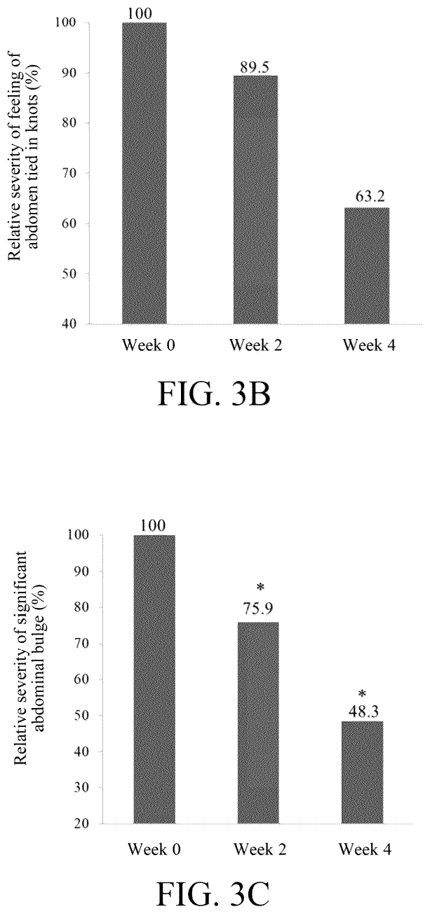

Referring to FIG. 3A, compared with week 0, the relative severity of difficulty in burping at week 2 was reduced to 75%. Compared with week 0, the relative severity of difficulty in burping at week 4 was reduced to 66.7%.

Referring to FIG. 3B, compared with week 0, the relative severity of feeling of abdomen tied in knots at week 2 was reduced to 89.5%. Compared with week 0, the relative severity of feeling of abdomen tied in knots at week 4 was reduced to 63.2%.

Referring to FIG. 3C, compared with week 0, the relative severity of significant abdominal bulge at week 2 was reduced to 75.9% (* represents a significant statistical difference compared with week 0 (p<0.05)). Compared with week 0, the relative severity of significant abdominal bulge at week 4 was reduced to 48.3% (* represents a significant statistical difference compared with week 0 (p<0.05)).

Based on this, after the subjects took the compound probiotics powder for four weeks, the relative severity of difficulty in burping, the relative severity of feeling of abdomen tied in knots, and the relative severity of significant abdominal bulge of the subjects decreased continuously. In other words, the bacterial species combination of the compound probiotics had the effects of alleviating difficulty in burping, feeling of abdomen tied in knots, and significant abdominal bulge of the human body.

3-2. Analysis on Compound Probiotics in Alleviating Defecation Discomfort and Increasing Defecation Frequency

The alleviation of the subjects was recorded with a questionnaire, and the comprehensive somatosensory assessment of defecation status (as in (1) below) and the records of defecation frequency (as in (2) below) were used to analyze the effects of the compound probiotics on the improvement of defecation status and promotion of defecation. Specifically, the survey on comprehensive somatosensory assessment of defecation status and defecation frequency was carried out with a questionnaire at week 0, week 2, and week 4 separately.

(1) Comprehensive Somatosensory Assessment of Defecation Status

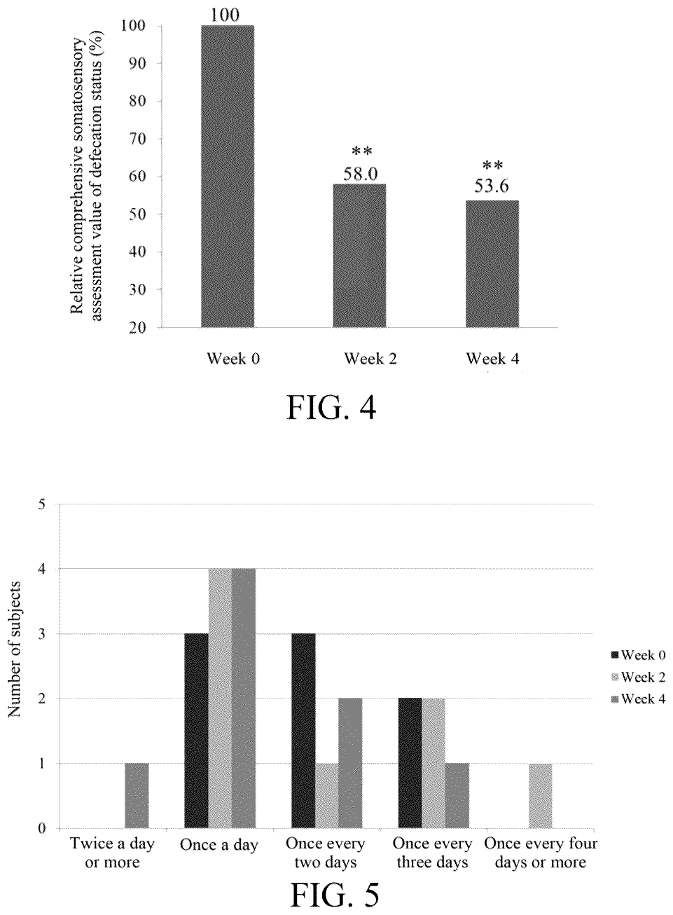

The questionnaire and scoring method are shown in Table 7. The left column of Table 7 lists various symptoms for assessment, including hard stool, unsmooth defecation, and difficulty in defecation when trying to defecate. The first row of Table 7 lists the scoring method, where a higher score indicates a more severe symptom. For example, if the subject suffered from severely unsmooth defecation, “very severe” (for 4 points) would be checked. On the contrary, if the subject did not suffer from unsmooth defecation, “none” (for 0 points) would be checked. In addition, the statistical results of Table 7 are shown in Table 8. The relative comprehensive somatosensory assessment values (%) of week 2 and week 4 were calculated respectively based on the mean value of week 0, as shown in FIG. 4.

TABLE 7
Questionnaire for comprehensive somatosensory
assessment of defecation status

| | Severity | | | | |
|---|---|---|---|---|---|
| | | | | | Very |
| | None | Mild | Medium | Severe | severe |
| Symptom | 0 point | 1 point | 2 points | 3 points | 4 points |

Hard stool
Low amount of feces
Unsmooth defecation
Painful defecation
Difficulty in
defecation when
trying to defecate

TABLE 8
Statistical results of comprehensive somatosensory
assessment of defecation status

| | Score of each item (sum of scores of 8 subjects) | | |
|---|---|---|---|
| Defecation symptom | Week 0 | Week 2 | Week 4 |
| Hard stool | 16 | 8 | 9 |
| Low amount of feces | 12 | 10 | 7 |
| Unsmooth defecation | 16 | 8 | 8 |
| Painful defecation | 10 | 6 | 6 |
| Difficulty in | 15 | 8 | 7 |
| defecation when | | | |
| trying to defecate | | | |
| Mean | 13.8 | 8.0 | 7.4 |
| % | 100.0 | 58.0 | 53.6 |

Referring to FIG. 4, compared with week 0, the relative comprehensive somatosensory assessment value of defecation status at week 2 was reduced to 58.0% (** represents a very significant statistical difference compared with week 0 (p<0.01)). In addition, compared with week 0, the relative comprehensive somatosensory assessment value of defecation status at week 4 was reduced to 53.6% (** represents a very significant statistical difference compared with week 0 (p<0.01)). Based on this, after the subjects took the compound probiotics powder for four weeks, the relative comprehensive somatosensory assessment value of defecation status of the subjects decreased continuously. In other words, the bacterial species combination of the compound probiotics had the effect of improving defecation status of the human body.

(2) Analysis on Defecation Frequency

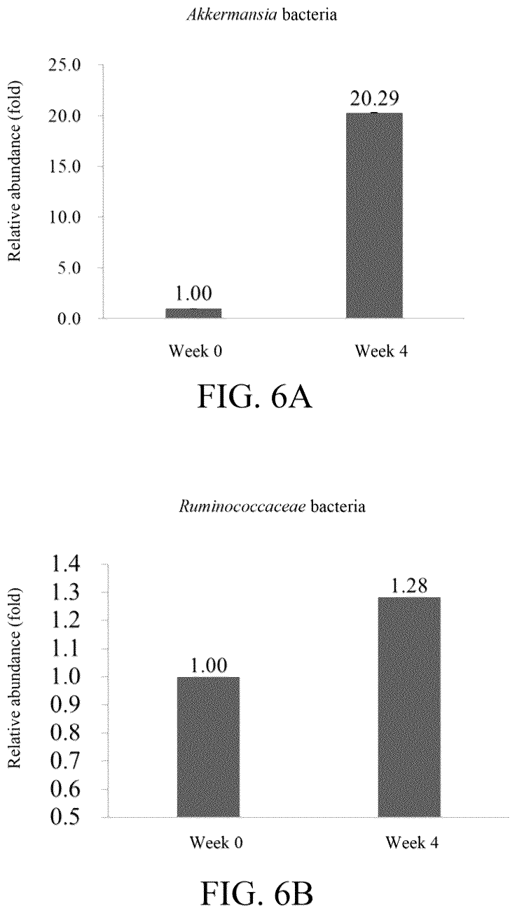

The questionnaire and scoring method are shown in Table 9. The first row of Table 9 lists the defecation frequency, including twice a day or more, once every two days, once every four days or more, and the like. In addition, the statistical results of Table 9 are shown in Table 10 and FIG. 5.

TABLE 9
Questionnaire for defecation frequency

| | | | | |
|---|---|---|---|---|
| Twice a | | | | Once every |
| day or | Once | Once every | Once every | four days |
| more | a day | two days | three days | or more |

Check
the most
suitable
option

TABLE 10
Statistical results of defecation frequency

| | Week 0 | Week 2 | Week 4 |
|---|---|---|---|
| | (number of | (number of | (number of |
| | the subjects) | the subjects) | the subjects) |
| Twice a day or | 0 | 0 | 1 |
| more | | | |
| Once a day | 3 | 4 | 4 |
| Once every two | 3 | 1 | 2 |
| days | | | |
| Once every three | 2 | 2 | 1 |
| days | | | |
| Once every four | 0 | 1 | 0 |
| days or more | | | |

Referring to FIG. 5 and Table 10, as a whole, with the advancement of the time axis of the human subject experiment (week 0 to week 4), the defecation frequency of the subjects increased. Specifically, the number of the subjects who defecated twice a day or more increased by one from week 4. The number of the subjects who defecated once a day was 3 at week 0, and increased to 4 at week 2, maintained at week 4. The number of the subjects with the other defecation frequencies (once every two days, once every three days, and once every four days or more) generally decreased at week 4 compared with week 0.

Based on this, after the subjects took the compound probiotics powder for four weeks, the defecation frequency of the subjects increased. In other words, the bacterial species combination of the compound probiotics had the effect of increasing defecation frequency of the human body.

3-3. Analysis on Compound Probiotics in Improving Gut Microbiota of Human Body

This example was to further determine the adjustment effect of the compound probiotics on the gut microbiota of the human body. At week 0 and week 4, the gut microbiota was detected. The feces of the subjects were collected as fecal samples, and the fecal samples were submitted to TOOLSBIOTECH Co., Ltd for analysis on gut microbiota.

The obtained fecal samples were subjected to extraction of genomic DNA. Next, the 16S rRNA sequence of the bacterial ribosome was subjected to sequence amplification in the V3-V4 region (from position 341 to position 806 of 16S rRNA sequence) using specific primers. The primers used for polymerase chain reaction are shown in Table 11. Then, the subsequent sequencing analysis was carried out by next-generation sequencing, and species annotation was carried out and the abundance index of each bacterial community was calculated. Based on the abundance index of week 0 (that is, the relative abundance of week 0 was 1), the abundance index of week 4 was converted into the relative abundance (fold) of week 4.

TABLE 11
Primer sequence

| Primer | | SEQ ID |
|---|---|---|
| name | Sequence | NO. |
| 341F | 5′-CCTACGGGNGG | 1 |
| | CWGCAG-3′ | |
| 806R | 5′-GACTACHVGGG | 2 |
| | TATCTAATCC-3′ | |

F: A: Adenine; C: Cytosine; G: Guanine; T: Thymine; W: Adenine/or Thymine; N: Adenine/Cytosine/Guanine/or Thymine.
R: A: Adenine; C: Cytosine; G: Guanine; T: Thymine; H: Adenine/Cytosine/or Thymine; V: Adenine/Guanine/or Cytosine.

In the gut microbiota, the probiotics include bacteria of Akkermansia and Ruminococcaceae. The pathogenic bacteria include bacteria of Bilophila and Veillonella.

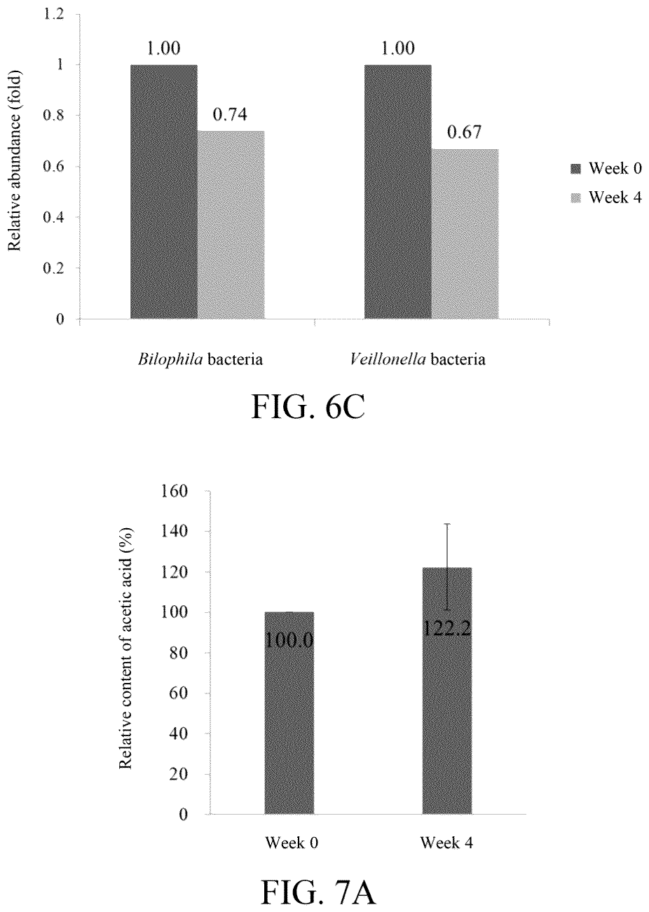

Referring to FIG. 6A, compared with week 0, the relative abundance of Akkermansia bacteria detected at week 4 was 20.29. In addition, referring to FIG. 6B, compared with week 0, the relative abundance of Ruminococcaceae bacteria detected at week 4 was 1.28.

Referring to FIG. 6C, compared with week 0, the relative abundance of Bilophila bacteria detected at week 4 was 0.74. In addition, compared with week 0, the relative abundance of Veillonella bacteria detected at week 4 was 0.67.

Therefore, after the subjects took the compound probiotics powder for four weeks, the gut microbiota of the subjects was adjusted. In other words, the bacterial species combination of the compound probiotics had the effect of improving gut microbiota. Specifically, the bacterial species combination of the compound probiotics could increase the abundance of probiotics (Akkermansia bacteria and Ruminococcaceae bacteria) and reduce the abundance of pathogenic bacteria (Bilophila bacteria and Veillonella bacteria).

3-4. Analysis on Content of SCFAs in the Human Gut

Metabolites are produced in the gut microbiota through fermentation. The metabolites include SCFAs. The SCFAs include acetic acid, propionic acid, butyric acid, isobutyric acid, and isovaleric acid.

This embodiment was to further determine the effect of the bacterial species combination of the compound probiotics on the improvement of gut microbiota. At week 0 and week 4, the contents of SCFAs of the subjects were detected respectively. Fecal samples having the same source as the test samples in Example 3-3 were used for testing.

Herein, the fecal samples were submitted to TOOLSBIOTECH Co., Ltd for detection of content of SCFAs. The detection process is not specifically described herein. After the detection, based on the content at week 0 (that is, the relative content at week 0 was 100%), the content at week 4 was converted into the relative content (%) at week 4.

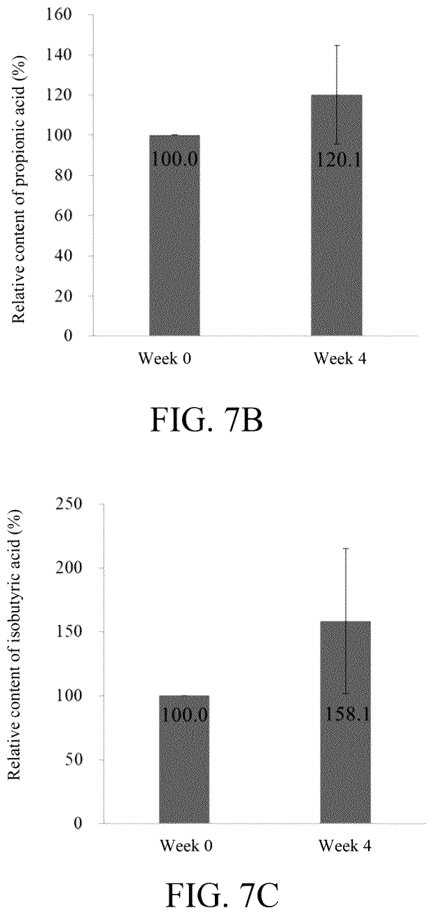

Referring to FIG. 7A, compared with week 0, the relative content of acetic acid at week 4 was 122.2%. In other words, the relative content of acetic acid at week 4 was increased by 22.2%.

Referring to FIG. 7B, compared with week 0, the relative content of propionic acid at week 4 was 120.1%. In other words, the relative content of propionic acid at week 4 was increased by 20.1%.

Referring to FIG. 7C, compared with week 0, the relative content of isobutyric acid at week 4 was 158.1%. In other words, the relative content of isobutyric acid at week 4 was increased by 58.1%.

Based on this, after the subjects took the compound probiotics powder for four weeks, the content of SCFAs in the gut of the subjects increased. In other words, the bacterial species combination of the compound probiotics had the effects of increasing the content of SCFAs in the gut and improving intestinal health care.

Based on the above, in the composition for improving gut microbiota according to any one of the embodiments, the bacterial species combination consisting of the specific bacterial species had the effect of improving gut microbiota of a subject. In some embodiments, the bacterial species combination consisting of the specific bacterial species had at least one of the following effects: reducing gas produced due to proliferation of E. coli, alleviating abdominal bloating, overcoming difficulty in burping, relieving a feeling of the abdomen tied in knots, alleviating significant abdominal bulge, improving defecation status, increasing defecation frequency, increasing the abundance of at least one of good bacteria, and increasing the content of SCFAs. Therefore, the bacterial species combination was suitable for preparing a composition for improving gut microbiota.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A composition for improving gut microbiota, comprising: a bacterial species combination consisting of Limosilactobacillus fermentum TCI275 with an accession number of BCRC 910940, Bifidobacterium animalis subsp. lactis TCI604 with an accession number of BCRC 910887, and Weizmannia coagulans TCI803 with an accession number of BCRC 910946.

2. The composition for improving gut microbiota according to claim 1, wherein the Limosilactobacillus fermentum TCI275, the Bifidobacterium animalis subsp. lactis TCI604, and the Weizmannia coagulans TCI803 are active strains.

3. The composition for improving gut microbiota according to claim 1, wherein the Limosilactobacillus fermentum TCI275, the Bifidobacterium animalis subsp. lactis TCI604, and the Weizmannia coagulans TCI803 are deactivated strains.

4. The composition for improving gut microbiota according to claim 1, comprising a food composition.

5. A method for improving gut microbiota of a subject in need thereof with a bacterial species combination, comprising administering to the subject an effective dose of a composition consisting of Limosilactobacillus fermentum TCI275 with an accession number of BCRC 910940, Bifidobacterium animalis subsp. lactis TCI604 with an accession number of BCRC 910887, and Weizmannia coagulans TCI803 with an accession number of BCRC 910946.

6. The method according to claim 5, wherein the gut microbiota of the subject in need thereof is improved by increasing an abundance of at least one of good bacteria.

7. The method according to claim 6, wherein the at least one of good bacteria is of Ruminococcaceae, Akkermansia, or a combination thereof.

8. The method according to claim 5, wherein the gut microbiota of the subject in need thereof is improved by increasing a content of short-chain fatty acids (SCFAs).

9. The method according to claim 5, wherein the composition for improving the gut microbiota of the subject in need thereof has an effect of overcoming difficulty in burping.

10. The method according to claim 5, wherein the composition for improving the gut microbiota of the subject in need thereof has an effect of alleviating flatulence.

11. The method according to claim 5, wherein the composition for improving the gut microbiota of the subject in need thereof has an effect of relieving a feeling of the abdomen tied in knots.

12. The method according to claim 5, wherein the Limosilactobacillus fermentum TCI275 and the Bifidobacterium animalis subsp. lactis TCI604 are isolated from human breast milk, and the Weizmannia coagulans TCI803 is isolated from orange peels.


SEQUENCE LISTING (2 sequences)

SEQ ID NO: 1 - DNA, 17 bp, synthetic construct
  source 1..17: mol_type = other DNA
  misc_difference 9: note = n is a, c, g, or t
  misc_difference 13: note = w is a or t
cctacgggnggcwgcag

SEQ ID NO: 2 - DNA, 21 bp, synthetic construct
  source 1..21: mol_type = other DNA
  misc_difference 7: note = h is a, c, or t
  misc_difference 8: note = v is a, g, or c
gactachvgggtatctaatcc